(19) United States
(12) Patent Application Publication
(10) Pub. No.: US 2003/0233138 A1
(43) Pub. Date: Dec. 18, 2003

(54) Concentration of divergent light from light emitting diodes into therapeutic light energy

(75) Inventor: Greg Spooner, San Francisco, CA (US)

Correspondence Address: Michael A. Stallman, STALLMAN & POLLOCK, LLP, 121 Spear Street, Suite 290, San Francisco, CA, 94105, US

(73) Assignee: Altus Medical, Inc., Burlingame, CA

(21) Appl. No.: 10/171,101

(22) Filed: Jun. 12, 2002

(51) Int. Cl. A61B 18/18

(52) U.S. Cl. 607/93; 607/88; 607/90

(57) ABSTRACT

The present invention generally provides improved therapeutic light sources and methods for their use. The invention also provides novel methods for fabricating therapeutic light sources. The present invention generally makes use of light emitting diodes (LEDs), and provides higher intensity therapeutic light than has previously been available with light emitting diode systems.

### BACKGROUND OF THE INVENTION

Field of the Invention

The present invention generally provides improved sources of therapeutic light for treatment for dermatological and other conditions, along with associated methods for fabricating and using therapeutic light sources.

A wide variety of light therapies have been developed over the last few decades to treat a number of conditions using light energy. Several of these therapies make use of light energy for treatment of dermatological conditions. For example, port-wine stain birthmarks and other subcutaneous vascular conditions may be treated by selectively heating the blood vessels with laser light energy. Similarly, selective heating of melanin with laser energy is now widely used for hair removal or epilation. Both of these therapies may be performed, for example, using a Nd:YAG laser having a wavelength of 1,064 nanometers such as that described in issued U.S. Pat. No. 6,383,176. Such laser-based treatments have been widely adopted, and are successfully treating large numbers of patients for a variety of dermatological and other conditions.

While generally successful, existing laser-based treatments are not without certain disadvantages. Specifically, known commercial laser therapy systems have often employed large, rather expensive lasers to generate sufficient therapeutic light energy. Many of these lasers require regular maintenance to provide the desired performance. Additionally, existing lasers are often relatively inflexible in the light wavelengths they produce. As different therapies benefit from different optical wavelengths, entirely separate laser systems are often required to perform different therapies.

More recently, both additional light-based therapies and alternative therapeutic light sources have been proposed. Laser light energy can be used for treatment of the retina, for reducing acne, and to improve the appearance of scars caused by trauma or prior surgeries. Along with standard lasers, proposed light sources include laser diodes, flashlamps, and the like. For lower energy application such as photo dynamic therapy in which light activates a drug for treatment of a target tissue, light emitting diodes (LEDs) have been proposed. While these alternative structures have significant cost advantages over conventional lasers, each has previously had significant disadvantages. When sufficient laser diodes are combined to generate therapeutic light energy, the total cost of the device can remain quite high. While flashlamps are very low in cost, the reflectors that typically collect the light and direct it to the skin are often precisely built and calibrated, as errors can product hot spots in the spatial energy distribution. Moreover, as the spectrum of light energy generated by lamps is quite broad, much of the total light energy may be either disadvantageous for a desired therapy or wasted by optical filters and the like. Hence, the structures associated with flash lamps can result in a larger and costly system, as well as decreasing reliability and efficiency, thereby mitigating the cost advantages of flash lamps over lasers. As many of the newly proposed light-based therapies are at least somewhat wave length specific, there remains a need for a low cost, wavelength-specific therapeutic light source.

### BRIEF SUMMARY OF THE INVENTION

The present invention generally provides improved therapeutic light sources and methods for their use. The invention also provides novel methods for fabricating therapeutic light sources. The present invention generally makes use of light emitting diodes (LEDs), and provides higher intensity therapeutic light than has previously been available with light emitting diode systems.

Unlike lasers (including conventional lasers and laser diodes), light emitting diodes generally generate divergent, non-coherent light. While the light energy from light emitting diodes generally extends throughout a significant band of wavelengths, most light emitting diodes are sufficiently wavelength-specific for targeted heating of a desired chromophore, targeted photochemical activation, targeted treatment depths, and the like. The highly divergent nature of the light generated from light emitting diodes makes concentration of the light power to therapeutic levels somewhat challenging. In many embodiments of the present invention, the light energy is concentrated by registering a plurality of optical waveguides (such as optical fibers) with an associated plurality of light emitting diodes. The light emitting diodes can be distributed throughout a considerable region. By bundling together the opposed ends of the light emitting diodes, and optionally by further concentrating light transmitted from bundled waveguide ends, sufficient light power may be transmitted to a target tissue to provide a light-based therapy, despite significant losses at the LED/waveguide interface. By including at least moderately efficient LED/waveguide light coupling structures and using a sufficient array of high-power light emitting diodes, a cost effective light therapy system is enabled despite the highly divergent nature of the generated light.

In a first aspect, the invention provides a therapeutic light source for treating a target tissue with a therapeutic light energy. The therapeutic light will often have a therapeutic light power density. The source comprises a plurality of LEDs, each LED transmitting divergent light. The LEDs are distributed across a first region. The divergent light across the first region has a first total light power density which is less than the therapeutic light power density. An optical train optically couples the LEDs with the target tissue. The optical train combines the divergent light and delivers the divergent light within a second region which is smaller than the first region so that the delivered divergent light has the therapeutic light power density.

In many embodiments, the therapeutic light energy at the target tissue will be significantly less than a total light power generated by the LEDs. This may be due at least in part to losses of the divergent light entering the optical train. Nonetheless, the optical train concentrates the divergent light sufficiently to overcome the optical train losses and increase the total light power density to provide the therapeutic light power density. In many cases, the optical train losses will comprise at least about half of the total light power generated by the LEDs. In some embodiments, overall optical coupling efficiency from the LEDs to the target may be less than 20%, in some cases being less than 10%, and occasionally being as low as 5%. Nonetheless, power density can be magnified from the first region of the light emitting diodes to the target tissue by one hundred times or more.

Preferably, the divergent light downstream of the optical train will have a power density of at least about 50 mW/cm². In many embodiments, the power density will be more than 1 W/cm², often being at least about 20 W/cm², and in some cases, being greater than 100 W/cm². These power densities will preferably be maintained throughout a treatment area of at least 1 mm², the treatment area optionally being defined by a light spot having the area of a 1.5 mm diameter circle, the treatment area optionally being about 10 mm² or more. To provide these total powers, there will often be at least about 50 light emitting diodes, optionally being 100 or more LEDs. Some and/or all of the LEDs may be supported by a common substrate, with the first region extending along one or more substrate and having an area of at least about 10 cm². An overall power density of the divergent light generated by the light emitting diodes within the first region may optionally be less than about 50 mW/cm². An amount of light energy generated by each LED may be at least about 20 mW of light power.

In many embodiments, the light emitting diodes will have a rated power, and will generate light at a rated power central wavelength. A circuit may overdrive the light emitting diodes beyond the rated power so that the divergent light has an overdriven central wavelength which is different than the rated power central wavelength. This overdriven central wavelength may selectively heat the target tissue. Overdriving of the light emitting diodes may be accomplished by using a short pulse duty cycle, and/or by accepting a short light emitting diode lifetime.

Optionally, the optical train may comprise a plurality of optical waveguides. Each waveguide may have a first end and a second end, at least a portion of the divergent light from each light emitting diode entering a first end of an associated waveguide. The first ends of the waveguides may be distributed adjacent the first region. The second ends of the optical waveguides may be bundled together within a second region which is smaller than the first region. Optionally, at least one lens surface may be disposed between each LED and the first end of the associated optical waveguide for directing the divergent light through the waveguide toward the second end. The lens may comprise a light concentrating lens such as a spherical lens, a bulb lens, an aspherical lens, a rod lens, or the like. In the exemplary embodiment, the lens surfaces comprise a spherical bulb end adjacent to the LED and a tapering condenser adjacent the first end of the optical waveguide.

A registration plate may support the first ends of the optical waveguides in alignment with the light emitting diodes. The registration plate may also support lenses concentrating light into the waveguides from between the LEDs and the first ends. Optical paths from the LEDs, through the lenses, and into the waveguides may have lateral tolerances (across the therapeutic light paths) and axial tolerances (along the therapeutic light paths), with the axial tolerances being looser than the lateral tolerances. Lateral positioning, for example, of a spherical bulb concentrating lens is preferably about 100μ or less, while the ends of the first optical fibers are axially positioned with a tolerance of about 300μ or less. In some embodiments, the lenses may be distributed in a two-dimensional array across an integrated lens structure.

In optional embodiments, the optical train may comprise an array of microlenses, each microlens directing light from at least one associated light emitting diode toward the target tissue. The microlenses may comprise cylindrical lenses, with the divergent light from each light emitting diode transmitted serially from a first cylindrical lens towards a second cylindrical lens, and from the second cylindrical lens toward the target tissue.

Optionally, an actively cooled surface may be disposed adjacent a light transmitting surface of the optical train for cooling a tissue surface adjacent the target tissue. The therapeutic light energy may have a central wavelength in a range from about 380 nm to about 800 μm, and a total delivered therapeutic light energy density may be sufficient for use as a therapy to mitigate acne.

In another aspect, the invention provides a therapeutic light source comprising a plurality of LEDs generating divergent light. A plurality of optical waveguides each have a first end and a second end. A plurality of light concentrators may be provided, and a registration substrate having a first plurality of positioning features and a second plurality of positioning features. The first positioning features each receive an LED. Each second position feature maintains registration between a first end of an optical waveguide and an associated LED with a light concentrator disposed therebetween so as to concentrate the divergent light from the light emitting diode into the waveguide. The second ends of the waveguides may be bundled together and transmit the divergent light.

The registration substrate may comprise at least one plate. The second positioning features may comprise a two-dimensional array of openings through the at least one plate for lateral positioning of the first ends of the optical waveguides across a plane of the at least one plate. The openings may laterally position the first ends of the optical waveguides, the light concentrators, and the LEDs with a lateral registration tolerance along the plane of the at least one plate. The openings may optionally define axial positioning surfaces for axially registering the light emitting diodes, the light concentrators, and the first ends of the optical waveguides along axes of the divergent light with an axial registration tolerance. The axial registration tolerance may be looser than the lateral registration tolerance.

The registration substrate may optionally comprise a first plate and a second plate. The openings through the first plate may laterally position the first ends of the optical waveguides, or the openings through the second plate may laterally position the light emitting diodes, with the light concentrators being disposed between the first and second plates. The plates may be positioned relative to each other by plate registration surfaces. The light concentrators may each comprise a body having a spherical lens surface adjacent the LED and an axially tapering optical condenser adjacent the optical waveguide.

In specific embodiments, adjacent light concentrators may be connected together to form a light concentrating array, and a light concentrating array may comprise a light transmitting material between concentrators. A combined light concentrator may be disposed between the second ends of optical waveguides and a target tissue. A combined light concentrator may direct light from the second ends of optical waveguides toward a target area of a target tissue. A target area may be smaller than an area of the second ends of the optical waveguides. A combined light concentrator may comprise a light condenser having a first surface adjacent to the optical waveguides and a second surface adjacent the target tissue. The second surface of the light condenser may be smaller than the first surface of the light condenser. A light source may comprise a cooling system capable of absorbing heat energy from a region adjacent the first ends of the optical fibers to accommodate divergent light from the LEDs which does not enter the waveguides.

In another aspect, the invention provides a method for fabricating a therapeutic light source. The method comprises registering an array of LEDs with an associated array of first optical waveguide ends so that a portion of divergent light from each LED enters an associated first end of an associated optical waveguide. The array has an array area. The optical waveguides downstream of the first ends are gathered together into a bundle having a bundle area which is less than the array area.

In another aspect, the invention provides a method for treating target tissue with a therapeutic light. The method comprises generating divergent light with a plurality of light emitting diodes. The LEDs are distributed within an LED region. At least a portion of the divergent light is concentrated with an optical train. The concentrated light from the optical train is transmitted to a target region of the target tissue. The target region is significantly smaller than the LED region, so that the concentrated light selectively heats and treats the target tissue.

### BRIEF DESCRIPTION OF THE DRAWINGS

FIG. 1 schematically illustrates a therapeutic light source according to the principles of the present invention, together with a method for its use.

FIG. 2 schematically illustrates concentration of light from a plurality of light emitting diodes distributed throughout a light generating region by directing at least a portion of the divergent light from the light emitting diodes into optical fibers, and by gathering ends of the optical fibers together into a light transmitting bundle having a size which is much smaller than that of the light generating region.

FIG. 3 schematically illustrates one form of divergent light emitted by a light emitting diode.

FIG. 4 schematically illustrates optical coupling of an optical fiber with a light emitting diode, and illustrates loss of a portion of divergent light.

FIGS. 5A-C schematically illustrate a registration substrate in the form of a plate supporting the light emitting diodes and a separate plate supporting the ends of the optical fibers in registration with the light emitting diodes.

FIGS. 6A and B illustrate a bulb lens for use as a light concentrator between a light emitting diode and an optical fiber, and illustrate light rays showing how the bulb lens enhances coupling of the divergent light from the light emitting diode.

FIG. 7 schematically illustrates an array of light emitting diodes maintained in registration with an associated array of optical fibers and bulb lenses by a registration substrate.

FIG. 8 schematically illustrates a second light concentrator in the form of a non-imaging optical condenser for increasing the light energy density between the bundled optical fibers and the target tissue.

FIG. 9 schematically illustrates an alternative second concentrator in the form of a spherical lens for increasing the light power density at the target tissue.

FIGS. 10A-C illustrate alternative optical concentrator suitable for use between the light emitting diodes and the optical fibers.

FIGS. 11A-F schematically illustrate the components and assembly of an array of light emitting diodes and associated optical fibers with light concentrator therebetween.

FIGS. 12A-D schematically illustrate components and assembly of an alternative light emitting diode and associated optical fiber registration system having an integrated light concentration structure.

FIG. 13 schematically illustrates a single condenser of a lens for concentrating light or from an array of light emitting diodes.

FIG. 14 schematically illustrates a multi-stage microlens array system for concentrating light from a plurality of light emitting diodes.

FIGS. 15A and B schematically illustrate overdriving of a light emitting diode using a pulsed driver system.

FIGS. 16A-C schematically illustrate an exemplary optical path from a light emitting diode, through an optical condenser, and into an optical fiber, together with computer light ray tracing analysis of the optical coupling efficiency.

FIGS. 17A and B schematically illustrate an experimental arrangement for determining light coupling efficiency as described herein.

### DETAILED DESCRIPTION OF THE INVENTION

The present invention generally provides devices and methods for collecting and concentrating light from multiple light emitting diodes (LEDs) for therapeutic purposes. The structures and methods of the present invention generally collect large quantities of radiant power and direct the radiant power into a sufficiently small area to achieve a desired therapeutic effect. In contrast, standard LED design approaches often optimize brightness (sometimes defined as the radiant power/area/solid angle) with reflectors or refractors that collect radiation over a relatively large area and direct it at a desired angle. By recognizing and accepting the relatively divergent nature of light emitted from LEDs, the present invention allows light density at a target plane to be sufficient for enabling LED-based light therapies which heat (often selectively) a target tissue, induce a photochemical change so as to effect a target treatment, and/or the like. For many applications, the therapeutic capability of a light source may be more greatly dependent on the light density at a target region than on the divergence of that light, particularly for dermal applications and other light therapies within a relatively short distance from a tissue surface, such as within 10 mm of the skin, and more commonly within 1 mm of the skin.

By enabling wavelength-specific light-based therapies using low cost LEDs, the systems and methods of the present invention will find applications for treatment of a wide variety of dermatological and other conditions. For example, the concentrating light energy may be used to selectively photo-destruct acne bacterium such as Propionibacterium Acnes. For such applications, the light energy will typically have an average irradiance of at least about 50 mW/cm² over a target treatment area of at least 1 mm². This allows an effective acne treatment (a fluence of about 100J/cm²) to be delivered to the target region in about 18 minutes. More preferably the light energy will have an irradiance of about 1.0 W/cm², ideally providing about 20 W/cm², often throughout a target treatment region of at least about 10 mm², ideally throughout a target region of at least about 100 mm². Alternative applications include treatment of port wine stains and other dermatological conditions, deepilation or hair removal, treatment of spider veins and tattoos, and the like. Such treatments often benefit from sufficient light energy for selectively heating of target tissues, often having power densities of at least about 20 W/cm², and many times having power densities of about 100 W/cm² or more.

Additional treatments benefited by the present invention include photocoagulation of vessels and other tissues, treatment of rosacea, hyperbilirubinemia, photodynamic therapies and photosensitizer assisted hair removal. An example of a photocoagulation treatment is treatment of telangiectasia, also referred to as spider veins. Photocoagulation of small blood vessels may be achieved with blue, green, yellow and red light. Blood vessels having a diameter of about 20 to 300 microns at lying at depths of tens and hundreds of microns below a skin surface may be treated with the present invention. For green light and yellow light having a cross sectional dimension of approximately 1 mm, photocoagulation is typically achieved with power densities of about 125 W/cm² and 100 W/cm² respectively. Blue light having a wavelength from about 400 to 500 nm is strongly absorbed by hemoglobin. Treatments using blue light may be achieved with power densities 20 times lower than are required for green and yellow wavelengths of light. For example, 50 mW of blue light optical power applied to a 1 mm spot may coagulate blood in a small volume. Treatments with blue light are typically localized to a shallower tissue penetration depth, for example a depth of tens of microns, and typically require less power than green or yellow light. Examples of tissues desirably treated with blue light include superficial skin vessels and tissues accessible with an endoscope. Examples of skin treatments include treatment of rosacea and telangiectasia.

Hyperbilirubinemia, also referred to as jaundice, may be treated by light energy having wavelengths from about 450 to 550 nm. Other wavelengths may be used to treat jaundice as described in co-pending U.S. Patent Application Serial No. 60/379,350, filed May 9, 2002, the full disclosure of which is incorporated herein by reference. For treatments with light energy having wavelengths between about 450 and 550 nm, power densities of 1 W/cm² substantially decrease treatment times. Treatments using these levels of power may benefit from epidermal cooling, for example active and efficient passive epidermal cooling, and infant jaundice may be treated with daily doses of applied light energy lasting minutes as opposed to several hours.

Tissue treatments with photodynamic therapy (PDT) may also benefit from the present invention. Systems and methods for treating tissue with photodynamic therapy are described in U.S. Pat. Nos. 6,269,818 to Lui et al., and 6,159,236 to Biel et al., the full disclosures of which are incorporated herein by reference. Many skin cancers are treated with photosensitizing agents, for example skin and esophageal cancers. A photosensitizing agent is applied to a tissue. During treatment, light energy excites a photosensitizing agent and generates free radicals, for example free radical oxygen species, that are toxic to tissue. In the case of cancer treatment, light energy is selectively applied to a cancer tissue. Examples of photosensitizing agents include, Photofrin™ and molecules having a porphyrin ring. Concentrated light available with the present invention permits rapid treatment, and cooling provided with embodiments of the present invention permits rapid treatment without over-heating tissue.

A tissue treatment for hair removal may also benefit from the present invention. A photosensitizing agent is applied to a skin having hair follicles. A photosensitizing agent may be sensitive to any of red, green, yellow and blue light. For a photosensitizer sensitive to red light, a red light energy may be applied to the skin. As light energy is applied to the skin, tissues having hair follicles are photochemically treated and the hair is easily removed. In some cases, hair follicles may be killed to permanently remove hair.

The output light energy from an LED typically comprises light energy having a band of wavelengths near a central emission wavelength. The spectrum of wavelengths of light energy emitted from a LED is often characterized as having a full width half maximum (FWHM) value based on the wavelengths at which the output energy intensity is half of a peak output intensity. Typical values of the FWHM for an emission spectrum of an LED ranges from about 5 nm to about 20 nm or more. A central wavelength of this emitted light energy can generally refer to the wavelength of a centroid of the emission spectrum. As used herein, the term light emitting diode (LED) excludes laser diodes generating coherent collimated light. Nonetheless, laser diodes may replace light emitting diodes in alternative embodiments of the present invention.

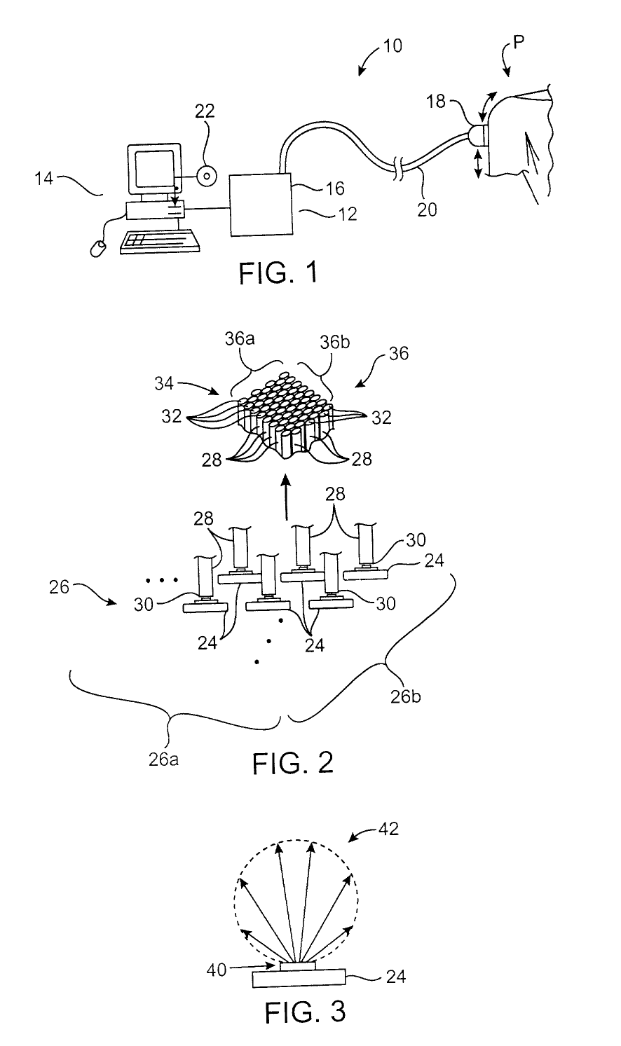

Referring now to FIG. 1, a light source system 10 for treating a target of a patient P generally includes a light source 12 coupled to a controller 14. Light source 12 includes a light generating assembly 16 coupled to a light application probe 18 by an optical transmission cable 20.

Controller 14 is schematically illustrated as a general purpose computer, and will typically include an input (such as a keyboard, mouse, Internet or other networking connection, wireless telemetry system, or the like), a display (such as a monitor, printer, or the like), and a processor. A tangible media 22 includes a computer program having instructions steps embodying one or more of the methods of the present invention, and may include data useful for operation of system 10. The tangible media may comprise a floppy disk, a CD, or other optical storage media, a RAM, non-volatile memory, an EEPROM, a hard disk accessible locally or over a network, or any of a wide variety of alternative forms. While a standard PC is schematically illustrated, a specialized processor may be integrated into other system components, or the like.

A light collection and concentration arrangement useful for light source 12 is schematically illustrated in FIG. 2. A plurality of light emitting diodes 24 are distributed throughout a first region within light generation assembly 16, the first region 26 schematically being illustrated with a length 26a and a width 26b. A plurality of optical waveguides 28 each have a proximal end 30 disposed adjacent an associated LED 24, and a distal end 32. The distal ends 32 of the optical waveguides 28 are gathered together into a bundle 34 having a second area 36, which is schematically illustrated with a length 36a and a width 36b. The area 36 of bundle 34 is much less than the LED light generation area 26, often being at least 10 times smaller, and generally being at least 100 times smaller.

Each LED will typically generate light with at least about 20 mW of optical power, preferably providing at least about 50 mW of output optical power and optionally generating at least about 200 mW of output optical power. The LEDs may be uniform so that each outputs light energy at a common wavelength. For example, each LED may output light energy having the same central wavelength. Alternatively, a plurality of different LEDs emitting light energy having different wavelengths may be used. Each LED may be individually removable and replaceable. In some embodiments, the LEDs may be replaceable in multiple units, for example, a subset of the array or the entire array of LEDs might be removable and replaceable for maintenance of the system 10. Optionally, some or all of the LEDs may be also removed and replaced with alternative LEDs having differing central wavelengths, thereby allowing system 10 to be used for a variety of differing light therapies. For example, for selective photo-destruction of acne bacterium, a first LED structure may be used to generate light with a wavelength at a local peak of Propionibacterium Acne. These first LEDs may be replaced with an array of LEDs generating a light suitable for photocoagulation of blood within the first millimeter of the dermal tissue so as to treat port wine stains. Hence, modular replaceability of one or multiple LEDs may be beneficial.

A wide variety of alternative LEDs structures might be employed. Optionally, LEDs 24 may each comprise a Microsemi Optomite UPVLED 400 having a central emission light energy wavelength of 410 nm. These LED devices are available from MICROSEMI, INC. of Irvine, Calif. In alternate embodiments, any LED having desired output optical power and light wavelengths may be used; for example, a Shark series part number OTL-395A-510-66-E multiple emitter LED having a central emission light energy wavelength of 395 nm and a rated output optical power of 250 mW, available from OPTO TECHNOLOGY INC. of Wheeling Ill.; a Lumileds Luxeon Star LXML-MM1C LED having a central emission light energy wavelength of 505 nm and a rated output optical power of 110 mW, available from LUMILEDS LIGHTING LLC of San Jose, Calif.; an Osram LV E67C LED having a central emission light energy wavelength of 503 nm and a rated output optical power of 7 mW, available from OSRAM OPTO SEMICONDUCTORS of San Jose, Calif.; an Osram LT E67C LED having a central light energy emission wavelength of 525 nm and a rated output optical power of 5 mW; a Lumileds Luxeon Star LXML-MM1C LED having a central light energy emission wavelength of 530 nm and a rated output optical power of 43 mW, available from LUMILEDS LIGHTING LLC; and a Shark series part number OTL-530A-5-10-66-E multiple emitter LED having a central light energy emission wavelength of 530 nm and a rated output optical power of 72 mW, available from OPTO TECHNOLOGY INC. Still further LEDs now under development (and optionally having powers beyond the above listed structures) may also be employed, particularly for treatment using light to selectively heat target tissues. The LEDs 24 may optionally be individually supported by an associated substrate, and in some cases, individually packaged with an associated end 30 of an optical waveguide 28. In alternative embodiments, the LEDs may be supported by a common substrate.

The optical waveguides may comprise optical fibers, light pipes, optical fiber bundles, or the like. Optical waveguides 28 will generally be coupled one-to-one with associated LEDs, the ends 30 of the optical waveguides having cross-sections or diameters sufficient that a significant fraction of light emitted by the LEDs can be coupled into the fiber. For example, optical waveguide 28 may comprise an optical fiber having a 1000 micron core such as that available commercially from Ceram Optec, of East Longmeadow, Mass. as a custom order OPTRAN WF 1000/1060 T optical fiber. Alternatively, an optical fiber having a 600 micron core may be used and is available commercially from Thorlabs of Newton N.J., model # FT-600-EMT. While a significant amount of light may be lost at the LED/fiber end coupling, the ability to bundle ends 32 of optical waveguides 28 into a bundle 36 having a size which is much less than the LED region 26 allows significant concentration of light power.

The optical power output by individual LEDs has been (and will likely continue to) increase significantly, but the overall optical power density from an array of LEDs remains somewhat limited. Specifically, the emitter size for each LED may be limited by thermal management considerations. This may make it difficult to increase a cross-sectional dimension of the emitter beyond a few hundred microns. Similarly, while it may appear advantageous to combine individual LED emitters into arrays of greater and greater density, the thermal management and power transmission design challenges (placement of wire bonds, heat sinks, and the like) may limit the number of emitters which may be supported on a common substrate per square unit of area.

By coupling each LED with an associated optical waveguide, and then bundling the optical waveguides together, individual emitters can be spread out on their supporting substrate as desirable for thermal management or other considerations. Hence, the optical power density of light transmitted from ends 32 at bundle 34 will preferably be at least 10 times the optical power density of the light generated by the LEDs distributed within diode region 26, power density at bundle 34 more preferably being at least about 20 times that of the total light power density throughout LED region 26, and ideally being at least 50 times. In the exemplary embodiments, ray tracing studies have indicated that the concentration of light power density form the LED region 26 may be 100 times or more, despite overall optical efficiencies between the LED and the target of as little as 5%.

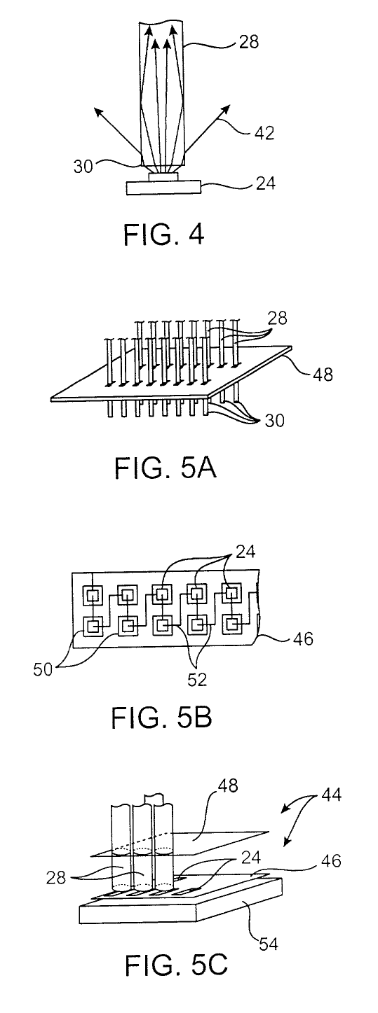

Referring now to FIG. 3, an individual LED 24 has a light emitting surface 40 which emits a highly divergent light 42. The divergent nature of light 42 may be Lambertian, having an intensity which varies as a cosine of the emission angle with respect to the emitter normal direction. A wide variety of alternative divergent light emitting characteristics may also be provided. As can be understood with reference to FIG. 4, coupling of the divergent light from LED 24 to optical waveguide 28 can be quite inefficient. As an example, a coupler having a 3 mm diameter LASFN9 glass ball lens and a 600 micron diameter multimode fiber waveguide centered over a domed, Lambertian emitter theoretically couples 33% of a total emitted LED irradiance into the waveguide. If the assembly is offset laterally by 600 microns, the coupling falls to 26%. An offset axially of 1000 microns decreases the coupling to 31%. A combination of a 600 micron lateral offset and a 1000 micron axial offset results in a coupling of 25%. Coupling efficiency is less sensitive to axial position than to lateral position. In general, axial position sensitivity will be less than lateral position sensitivity, and sensitivity to lateral and axial positions will be related to dimensions of the coupler and LED. Hence, accurate registration of optical waveguides 28 with LEDs 24 significantly improves total power density.

Referring now to FIGS. 5A-C, efficient and cost-effective coupling between large numbers of LEDs 24 and associated ends 30 of optical waveguides 28 may be facilitated by use of a registration substrate 44, the registration substrate here comprising a plurality of registration plates 46, 48. LEDs 24 may be pre-positioned and fixed within a translational and axial tolerance to a first or LED registration plate 46. Optical waveguides 28 may similarly be pre-positioned and fixed relative to a second or fiber registration plate 48. The pre-assembled plates may then be registered with each other using inter-plate registration surfaces (not shown in FIGS. 5A-C) so as to provide effective optical coupling between the emitters of LEDs 24 and their associated waveguides 28. The register structures may then be packaged together within a housing to form light generating assembly 16 (see FIG. 1). As illustrated in FIG. 5B, the LED registration plate 46 may provide individual heat spreaders 50 for each LED 24, along with electrical leads 52 for powering of the LEDs. Active or passive cooling elements 54 may also be included within the package, the cooling optionally comprising heat sink materials, liquid cooling channels for water, ethylene glycol, liquid nitrogen or the like, thermo-electric cooling elements, or the like.

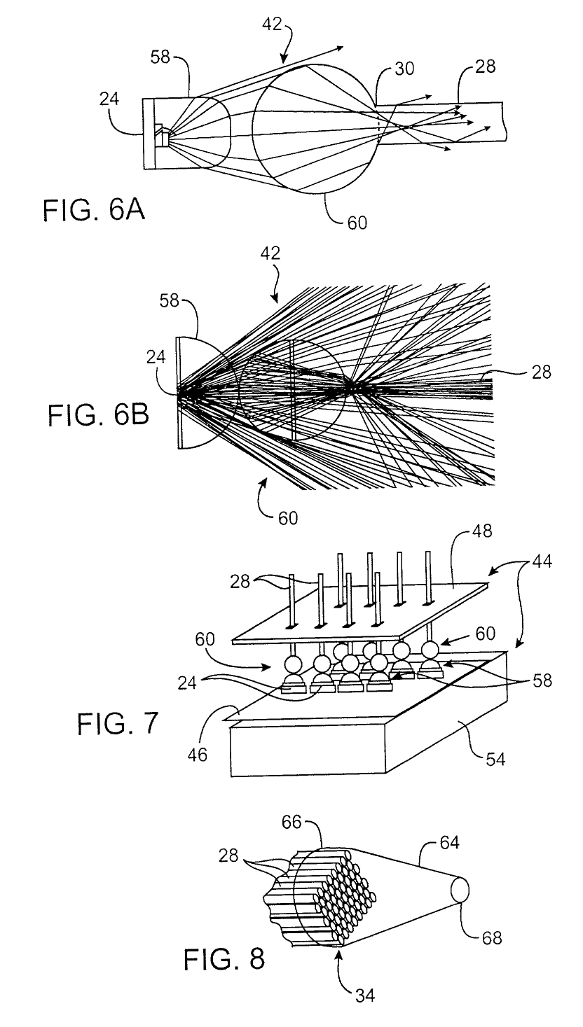

Referring now to FIGS. 6A and 6B, LEDs are often packaged with dome lenses 58 for increasing brightness. Regardless whether or not LED 24 has a dome lens 28, a light concentrating ball lens 60 (or other light concentrator) may be disposed between LED 24 and end 30 of optical fiber 28 so as to increase optical coupling of divergent light 42. The improved capture of the divergent light 42 by optical waveguide 28 is schematically illustrated in FIG. 6A and can also be understood with reference to the ZEMAXTM ray trace of a Lambertian LED 24 with a dome lens 58, together with ball lens 60 disposed between dome lens 58 and optical waveguide 28, as shown in FIG. 6B. ZEMAX™ ray trace software is available from Focus Software of Tucson, Ariz.

Referring now to FIG. 7, a registration structure 44 similar to that described above with reference to FIG. 5C includes LEDs having dome lenses 58 and ball lenses 60 for concentrating light from the emitters of the LEDs into the optical waveguides 28. By using, for example, an array with 80 commercially available high-flux LEDs (such as those sold by LUMILEDS under the tradename Luxeon Star LXHL with Lambertian dome) coupled to a 1000 μ core fiber by 6 mm ball lenses (such as Schott high index glass LASFN 9 ball lenses available from Edmund Scientific of Barrington, N.J.), therapeutic light power densities may be delivered. Each LED produces about 100 mW of green light at an emitting aperture power density of about 0.15 W/cm² at maximum pulsed current operation. The coupled light, when concentrated by a secondary light concentrator such as condenser 64, can deliver 0.15 W in a relatively small spot having a diameter of about 1.5 mm, thereby producing a power density at the target tissue of more than 20 W/cm².

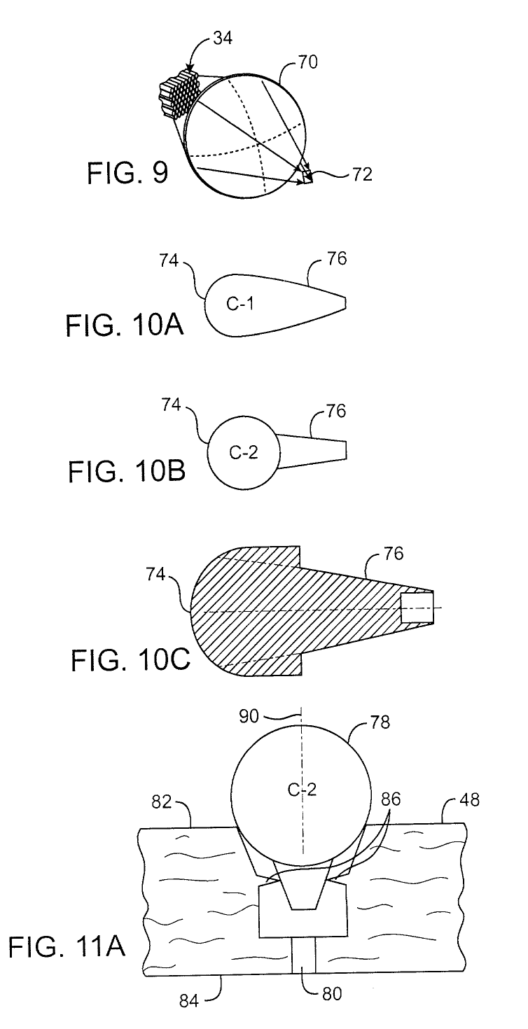

Secondary light concentrators are illustrated in more detail in FIGS. 8 and 9. Non-imaging light condenser 64 generally concentrates a light transmitted from bundled ends 34 of waveguides 28. Light concentrator 64 typically has a first end 66 near bundle 34 opposed to a second end 68, with the opposed light transmitting end 68 having an area which is significantly smaller than that of light receiving end 66 near the optical waveguides. Such a light pipe optic concentrator may again involve significant losses, but nonetheless provides increased power densities. Alternatively, a non imaging condenser may comprise several fibers having a decreasing diameter. For example, several fibers may have a decreasing cross sectional diameter as light propagates toward an end of a probe, and the fibers may terminate near a transmitting end of a probe.

The light emerging at the light transmitting surface 68 may be highly divergent. Nonetheless, as noted above, the divergence and brightness of the output light may be secondary to the power density and total power of therapeutic light, particularly when therapeutic light energy is to be applied near light transmitting end 68 of light concentrator 64. Optionally, cooling of the tissue targeted for treatment may be effected prior to, after, and/or during application of the therapeutic light energy, optionally using a tissue cooling surface such as that described in U.S. Pat. No. 6,383,176. In some alternatives, cooling fluid may flow through light concentrator 64 for thermal cooling via light transmitting surface 68. An alternative light condenser 70, here comprising a spherical lens, is illustrated in FIG. 9. Regardless of the specific light concentrating structure used, a target tissue region 72 will generally be smaller than a size of the bundle 34 when a secondary light concentrator is included in light system 10.

Referring now to FIGS. 10A-C, alternative light concentrators 78 for improving coupling efficiency between a light emitting diode and an optical waveguide are shown. Each includes a spherical or ball lens surface to be oriented toward a corresponding emitter of a LED 24, and tapering light condenser 76 for concentrating light into an associated optical waveguide 28. The light condenser illustrated in FIG. 10C (shown here in cross-section) can be machined from polycarbonate, and may be vapor-honed using an atmosphere of methylethyl-ketone to smooth the surfaces. Index matching between a cleaved fiber (such as a 1 mm fiber) and the light concentrator may improve coupling efficiency, as may avoiding strain of the cleaved fiber end. Index matching may be provided by mineral oil or a suitable optical adhesive. Couplers 78 may optionally comprise glass, plastics, combinations of glass and plastics, and will often include a high index material when spherical surfaces are employed. Fiber registration plate 48 may comprise a wide variety of materials such as silicon, polymers such as those employed for printed circuit boards and other plastics, metals such as Kovar. Still further alternative structures may be employed, including integrated optical waveguide substrate and optical transmission structures that have recently been developed for fiber optic communications, and the like.

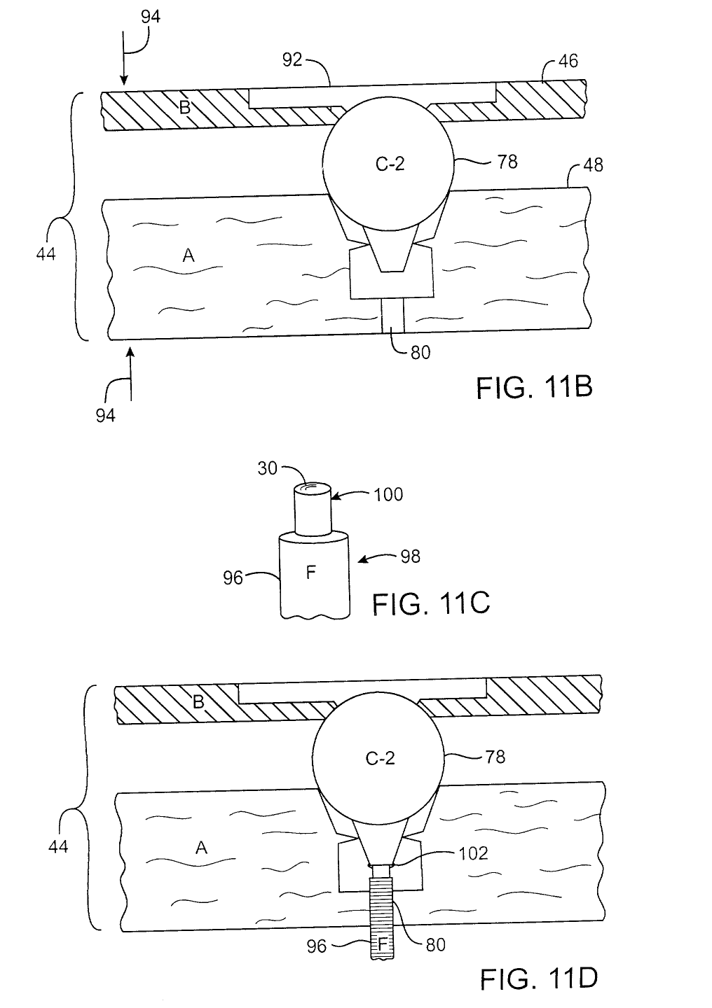

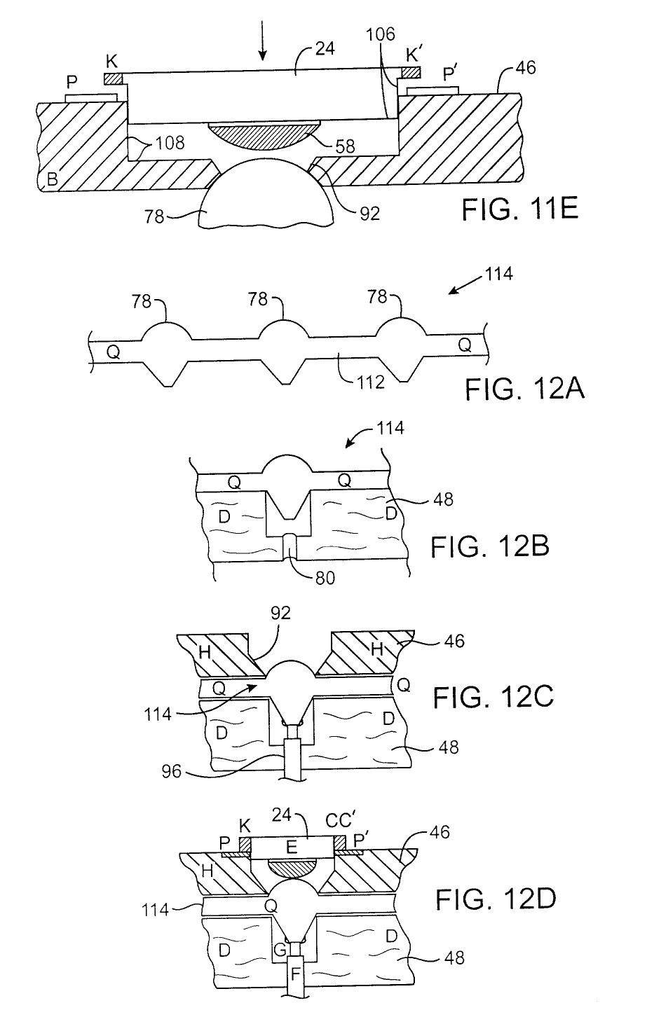

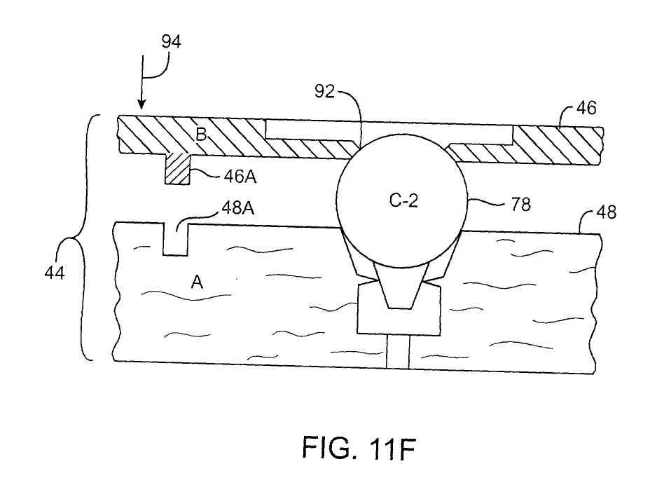

Referring now to FIGS. 11A-D, an exemplary light generation assembly, registration substrate and fabrication method for light generating system 16 will be described. While only a single registered LED and optical fiber are illustrated in these figures, these structures and methods are particularly useful when used for registration of a plurality of LEDs and associated optical waveguides, as schematically illustrated in FIG. 7. As seen in the more detailed schematic cross-sectional illustration of FIG. 11A, fiber-supporting registration plate 48 includes a plurality of openings 80 between a first major surface 82 and a second major surface 84. Openings 80 include positioning surfaces 86 which receive concentrator 78, and which position the concentrator relative to the fiber registration plate 48. Optionally, surfaces 86 may position concentrator 78 axially relative to an axis 90 of an eventual optical path, laterally relative to axis 90, and/or orientationally, for example, so as to maintain coaxial alignment between the axis 90 of the optical path and a corresponding axis of concentrator 78. In the exemplary fiber registration plate 48 (schematically illustrated in FIG. 1A) the concentrator 78 may be positioned by simply placing and/or dropping the concentrators (in roughly the correct orientation) into openings 80. Suitable openings may be fabricated using a variety of techniques, including microelectromechanical structure (MEMS) technologies which were largely developed for the electronics fabrication industry, and which may employ photolithography, etching, selective deposition, and the like to produce highly accurate and repeatable registration plate features.

Referring now to FIG. 11B, LED registration plate 46 may be registered with fiber registration plate 48 by engagement between an opening 92 formed in LED registration plate 46 and a surface of coupler 78, or by engagement between corresponding surfaces of the two registration plates (as illustrated below). The combined registration plates together define a registration substrate 44, and can clamp the couplers accurately into alignment with openings 80 and 92, as schematically illustrated by arrows 94. As shown in isolation in FIG. 11C, an optical fiber 96 includes a jacket 98 and a core 100. To improve optical coupling efficiency, it may be beneficial to polish or cleave optical fiber 96, and optical adhesive may be deposited on end 30 of the optical fiber either individually, or by, for example, dipping a jig supporting an array of fibers 96 into a suitable adhesive 102. By inserting fiber 96 into opening 80 and curing adhesive 102, the optical fibers and their associated couplers 78 may be registered with registration substrate 44, as illustrated in FIG. 11D.

As shown in FIG. 11E, light emitting diode 24 may be registered with the remaining components of the assembly by fitting engagement between corresponding surfaces of the LED 106 and surfaces 108 of opening 92 through LED registration plate 46. Once again, registration of the LED (and its emitter surface) relative to the optical path should be within axial, lateral and/or orientational tolerances so as to provide sufficient optical coupling. Nonetheless, significant losses may still be noted at the LED/fiber interface due to the highly divergent nature of the generated light. Once LED 24 is slid into position within opening 92, contacts K, K′ of the LED may electrically couple the LED with corresponding contacts P, P′ (such as solder pads, photolithographically deposited leads, or the like) on LED registration plate 46.

Referring now to FIG. 11F, a registration substrate 44 may include an LED registration plate 46 and a fiber registration plate 48 as described above. An opening 48A formed in a surface of fiber registration plate 48 receives an extrusion 46A formed in a surface of LED registration plate 46. As illustrated in FIG. 11F, coupling between opening 48A and extrusion 46A may improve alignment between LED registration plate 46 and fiber registration plate 48. Positions of openings formed in a surface of fiber registration plate 48 and extrusions formed in surface of LED registration plate 46 determine relative positioning of fiber registration plate 48 with LED registration plate 46. Alternatively, openings may be formed in LED registration plate 46 and extrusions may be formed in fiber registration plate 48. As many coupled openings and extrusions as needed may be provided. For example, large plates may include at least 4 coupled openings and extrusions located centrally and peripherally on plates 46 and 48.

A structure and method similar to that described above regarding FIGS. 11A-F can be understood with reference to FIGS. 12A-D. However, in this embodiment, couplers 78 are interconnected by light coupling material 112 so as to form a integrated coupler array structure 114, as illustrated in FIG. 12A. Couplers 78 are positioned initially and/or at least in part by engagement between integrated coupler plate 114 and fiber registration plate 48, as shown in FIG. 12B. Laterally engaging the surfaces of the integrated concentrator plate and fiber and LED registration plates 48, 46 of registration substrate 44 may help maintain axial alignment of coupler 78 between LEDs 24 and their associated optical fibers 96. The remaining assembly steps are similar to those described above regarding FIGS. 11A-F.

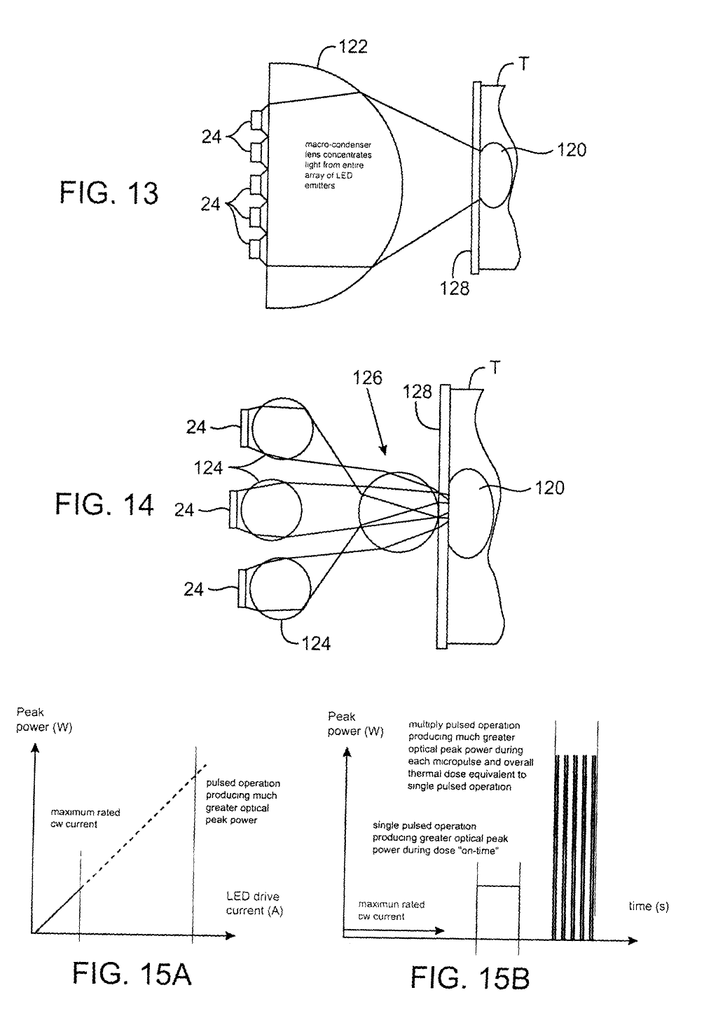

Referring now to FIGS. 13 and 14, still further alternative light concentration structures might optionally be used to couple the light output of LEDs and concentrate the light to therapeutic fluences. It may be difficult to produce one-to-one or smaller imaging using a single or compound lens system, as F-numbers greater than 1 are not typically available via an air-glass design using a single or compound lens system. Nonetheless, a target tissue 120 may be treated by concentrating a light from an array of LEDs 24 using a single monolithic condenser lens 122 as illustrated in FIG. 13, or an array of microlenses 124 as illustrated in FIG. 14. Array 124 may be assembled from individual components such as optical fibers, or may be made from inexpensively molded or machined monolithic microlens arrays. In either case, cylindrical lenses 122 and 124 are schematically illustrated for concentration of the light energy from an array of LEDs 24. A second stage of condensing optics 126 is also schematically illustrated in FIG. 14 as a cylindrical lens. In both cases, a window 128 provides an interface to tissue T, and may provide cooling of target tissue 12 via the flow of cooling fluid or the like.

Referring now to FIGS. 15A and 15B, the coherence and brightness associated with laser therapy treatments are quickly lost or significantly degraded when the light energy is used in high scattering tissues such as the dermis. While LEDs are characterized by wide divergence, relatively lower brightness, and lower coherence than lasers, they may be used for a variety of therapeutic treatments so long as the medically effective fluence levels can be obtained. Optionally, these desired fluence levels may be obtained at least in part by employing a pulsed operation in which the LEDs are overdriven to produce output powers many times (as much as 10 times) more than the rated power for standard long-life continuous output operation. A significantly decreased lifetime of the LEDs may be overcome by designing an application structure so that LEDs are not required to last prolonged periods of time and may be replaced, disposed of, or included in a consumable subassembly.

Pulsing of the drive circuitry may provide bursts of very high peak power or “micropulses” may be used to produce the appropriate thermal doses. One little-recognized aspect of overdriving is that it may tend to “blue” the wavelength of energy generated by the LED. While a minor shift of the wavelength of generated light toward the ultraviolet by some overdriven LEDs may not vary their effectiveness, the wavelength-specific chromophores and interactions in some therapies may make it beneficial to select an LED structure having an appropriate center wavelength during overdriven (rather than maximum rated continuous) operation. This aspect of the present invention, along with treatments which might be effected using structures such as those described herein for mitigation of acne, are more fully described in co-pending U.S. Provisional Patent Application No. 60/379,350, filed on May 9, 2002, and entitled “System and Methodfor Treating Exposed Tissue with Light Emitting Diodes” (Attorney Docket No. 019593-00110US), the full disclosure of which is incorporated herein by reference.

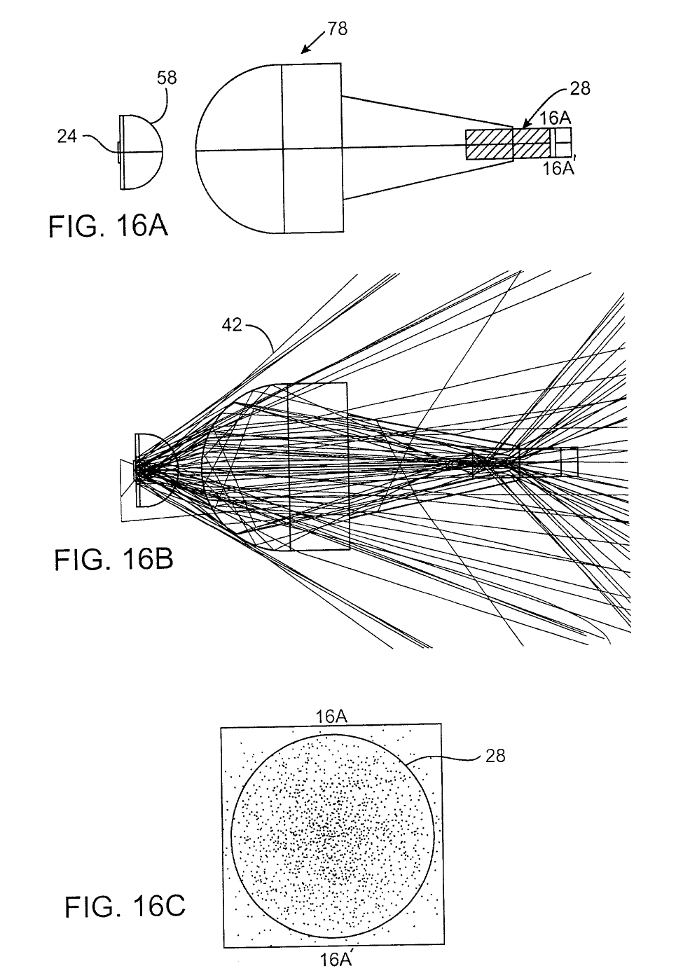

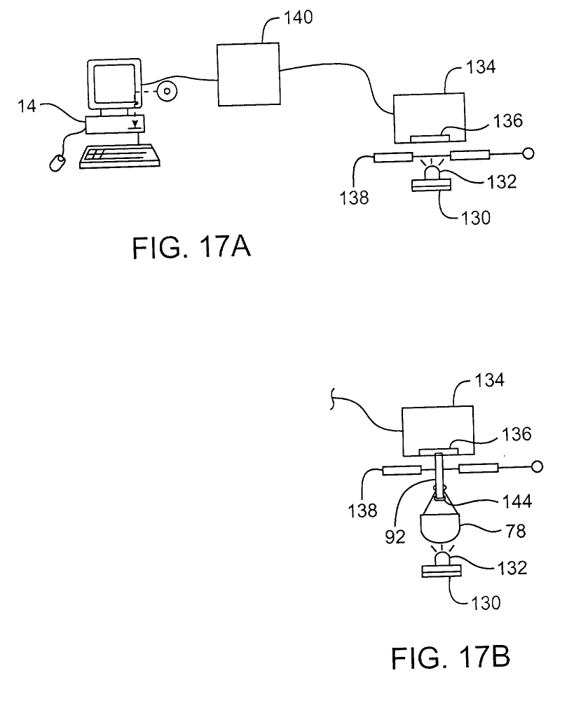

FIGS. 16A through 17B illustrate computer modeling and corresponding experimental results showing coupling between LEDs 24 and associated optical waveguides 28 using light concentrators 78. The computer modeled results of FIGS. 16A-C first show the LED/waveguide interface optics in isolation (in FIG. 16A). FIG. 16B illustrates graphically the divergent light 42 generated by a LED 24, and shows a computer generated plot of rays as they transit the interface. Despite the significant loss of light at the interface, a coupling efficiency based on the computer model was estimated to be roughly about 31%. FIG. 16C illustrates the density of ray tracings passing through waveguide 28 at section 16A, 16A′, as seen in FIG. 16A.

Corresponding experimental results were obtained using the arrangement illustrated schematically in FIGS. 17A and 17B. Referring first to FIG. 17A, an Osram LT E67C light emitting diode 130 having a dome lens 132 with a diameter of about 2.5 mm was first tested to determine a rough total light power output. An integrating sphere 134 was positioned with a light inlet 136 laterally aligned with LED 130, with the LED advanced axially as close as possible to the integration sphere light inlet with a variable iris diaphragm 138 disposed therebetween. Light from integrating sphere 134 was coupled to a USB 2000 spectrometer 140, and the measured light output was analyzed by a controller 14 resulting in a measured power output of about 1.0 mW. A USB 2000 spectrometer is available from Ocean Optics, Inc. of Dunedin, Fla. This total output measurement was taken with iris diaphragm 138 opened to a size at least corresponding to opening 136 into the integrating sphere 134. The emitter surface of LED 130 was measured using a STM microscope, indicating an apparent lateral cross-sectional size (relative to the optical path) of about 0.75 mm.

The experimental arrangement of FIG. 17A was modified to that shown in FIG. 17B for determining the coupling efficiency between an optical waveguide and an LED. In this experiment, a concentrator 78 having the form illustrated in FIG. 10C was fabricated as described with reference to that Fig. from polycarbonate and vapor-honed. This concentrator was then coupled to an optical waveguide in the form of a 2 cm length of 1000 micron diameter silica optical fiber as described above. The optical fiber end adjacent concentrator 78 was cleaved and mineral oil 144 was disposed between the optical fiber and the concentrator for index matching. Iris diaphragm 138 was closed about fiber 92 so as to inhibit transmission of light other than that transmitted by optical fiber 92 into integrating sphere 134. The coupler was held in a 3-axis translation stage to allow for optimization of the coupler position with respect to the LED. Positioning of the coupler could be controlled to within 10 microns. Additionally, the tilt of the coupler was adjusted manually. The total amount of light energy measured by spectrometer 140 was about 0.29 mW, indicating an overall coupling efficiency of about 29%. It should be noted that not all of the light generated by the divergent LED structure may have been measured by the arrangement illustrated in FIG. 17A, nonetheless, the agreement between the modeling results helps verify that therapeutic light power densities may be generated and concentrated using the structures and methods described herein. The experimental setup illustrated in FIGS. 17A and 17B has shown that index matching and avoiding undue strain at the concentrator/fiber interface significantly improves coupling results. Additionally, coupling of the larger high-power emitting surfaces of the new high output LEDs may somewhat decrease overall coupling efficiency when relatively smaller optical waveguides are used to transmit the coupled light. Nonetheless, as quite reasonable coupling efficiencies can be provided, and as light concentration from the relatively widely dispersed LEDs to the bundled optical fiber ends can provide light concentration ratios of greater than 10 and in some cases being greater than 100, and possibly being greater than 200 times, therapeutic light power densities may now be available from low-cost LED structures.

While the exemplary embodiments of the present invention have been described in some detail, by way of example and for clarity of understanding, a variety changes, adaptations, modifications, and substitutions will be obvious to those of skill in the art. Hence, the scope of the present invention is limited solely by the appended claims.

What is claimed is:

1. A therapeutic light source for treating a target tissue with a therapeutic light energy having a therapeutic light power density, the source comprising:
a plurality of LEDs, each LED transmitting divergent light, the LEDs distributed across a first region, the divergent light across the first region having a first total light power density less than the therapeutic light energy; and
an optical train optically coupling the LEDs with the target tissue, the optical train combining the divergent light and delivering the divergent light within a second region smaller than the first region so that the delivered divergent light has the therapeutic light power density.

2. The therapeutic light source of claim 1, wherein therapeutic light power at the target tissue is significantly less than a total light power generated by the LEDs due at least in part to losses of the divergent light entering the optical train, and wherein the optical train concentrates the divergent light sufficiently to overcome the optical train losses and increase the total light power density from the first light power density to the therapeutic light power density.

3. The therapeutic light source of claim 2, wherein the optical train losses comprise at least about half of the total light power generated by the LEDs.

4. The therapeutic light source of claim 1, wherein the divergent light downstream of the optical train has an power density of at least about 1 W/cm².

5. The therapeutic light source of claim 4, where the divergent light downstream of the second ends has the power density throughout an area of at least about 1.0 mm².

6. The therapeutic light source of claim 4, wherein at least some of the LEDs are supported by a substrate, the first region extending along the at least one substrate and having an area of at least about 10 cm².

7. The therapeutic light source of claim 6, wherein an overall power density of the divergent light generated within the first region is less than about 50 mW/cm².

8. The therapeutic light source of claim 4 wherein each LED generates at least about 20 mW of light power.

9. The therapeutic light source of claim 8, the LEDs having a rated power and generating light at a rated power central wavelength, further comprising a circuit overdriving the LEDs beyond the rated power so that the divergent light has an overdriven central wavelength is different than the rated power central wavelengths, the overdriven central wavelength selectively heating the target tissue.

10. The therapeutic light source of claim 1, wherein the optical train comprises a plurality of optical waveguides, each waveguide having a first end and a second end, at least a portion of the divergent light from each LED entering a first end of an associated waveguide, the first ends of the waveguides being distributed adjacent the first region, the second ends of the optical waveguides being bundled together within a second region smaller than the first region.

11. The therapeutic light source of claim 10, further comprising a plurality of lens surfaces, at least one of the lens surfaces being disposed betweeri each LED and the first end of the associated optical waveguide for directing the divergent light through the waveguide toward the second ends.

12. The therapeutic light source of claim 10, wherein each lens surface comprises a spherical lens, and further comprising a light condenser decreasing in cross-section from the spherical lens to the optical waveguide.

13. The therapeutic light source of claim 10, further comprising a registration plate supporting the first ends of the optical waveguides in alignment with the LEDs.

14. The therapeutic light source of claim 13, wherein the registration plate supports a plurality of lenses, the registration plate maintaining optical paths from each LED to an associated optical waveguide with the lenses concentrating the light into the waveguide.

15. The therapeutic light source of claim 14, the optical paths having lateral tolerances laterally oriented across the light paths and axial tolerances axially oriented along the optical paths, the axial tolerances being looser than the lateral tolerances.

16. The therapeutic light source of claim 14, wherein the plurality of lenses are distributed in a two dimensional array across an integrated lens structure.

17. The therapeutic light source of claim 1, wherein the optical train comprises an array of microlenses, each microlens directing light from at least one associated LED toward the target tissue.

18. The therapeutic light source of claim 17, wherein the microlenses comprise cylindrical lenses, and wherein the divergent light from each LED is transmitted serially from a first cylindrical lens toward a second cylindrical lens, and from the second cylindrical lens toward the target tissue.

19. The therapeutic light source of claim 1, further comprising an actively cooled surface disposed adjacent a light transmitting surface of the optical train for cooling a tissue surface adjacent the target tissue.

20. The therapeutic light source of claim 1, wherein the therapeutic light energy has a central wavelength in a range from about 380 nm to about 800 nm, and wherein the therapeutic light power density is sufficient to mitigate acne of the target tissue.

21. A therapeutic light source comprising:
a plurality of LEDs, each LED generating divergent light;
a plurality of optical waveguides, each waveguide having a first end and a second end;
a plurality of light concentrators;
a registration substrate having a first plurality of positioning features and a second plurality of positioning features, the first positioning features each receiving an LED, each second positioning feature maintaining registration between a first end of an optical waveguide and an associated LED with a light concentrator disposed therebetween so as to concentrate the divergent light from the LED into the waveguide;
the second ends of the waveguides being bundled together and transmitting the divergent light.

22. The therapeutic light source of claim 21, wherein the registration substrate comprises at least one plate, the second positioning features comprising a two-dimensional array of openings through the at least one plate for lateral positioning of the first ends of the optical waveguides across a plane of the at least one plate.

23. The therapeutic light source of claim 22, wherein the openings laterally position the first ends of the optical waveguides, the light concentrators, and the LEDs with a lateral registration tolerance along the plane of the at least one plate, and wherein the openings define axial positioning surfaces for axially registering the LEDs, the light concentrators, and the first ends of the optical waveguides along axes of the divergent light with an axial registration tolerance, the axial registration tolerance being looser than the lateral registration tolerance.

24. The therapeutic light source of claim 22, wherein the at least one registration plate of the registration substrate comprises a first plate and a second plate, the openings through the first plate laterally positioning the first ends of the optical waveguides, the openings through the second plate laterally positioning the LEDs, the light concentrators being disposed between the first and second plates.

25. The therapeutic light source of claim 24, wherein the light concentrators each comprise a body having a spherical lens surface adjacent the LED and an axially tapering optical condenser adjacent the optical waveguide.

26. The therapeutic light source of claim 21, wherein the adjacent light concentrators are connected together to form a light concentrating array, the light concentrating array comprising light transmitting material between the concentrators.

27. The therapeutic light source of claim 21, further comprising a combined light concentrator disposed between the second ends of the optical waveguides and the target tissue, the combined light concentrator directing light from the second ends of the optical waveguides toward a target area of the target tissue, the target area being smaller than an area of the second ends of the optical waveguides.

28. The therapeutic light source of claim 26, wherein the combined light concentrator comprises a light condenser having a first surface adjacent to the optical waveguides and a second surface adjacent the target tissue, the second surface of the light condenser being smaller than the first surface of the light condenser.

29. The therapeutic light source of claim 26, further comprising a cooling system capable of absorbing heat energy from a region adjacent the first ends of the optical fibers to accommodate divergent light from the LEDs which does not enter the waveguide.

30. A method for fabricating a therapeutic light source, the method comprising:
registering an array of LEDs with an associated array of first optical waveguide ends so that a portion of divergent light from each LED enters an associated first end of an associated optical waveguide, the array having an array area;
gathering together the optical waveguides downstream of the first ends into a bundle having a bundle area less than the array area.

31. A method for treating a target tissue with therapeutic light, the method comprising:
generating divergent light with a plurality of LEDs, the LEDs distributed within an LED region;
concentrating at least a portion of the divergent with an optical train; and
transmitting the concentrated light from the optical train to a target region of a target tissue, the target region being significantly smaller than the LED region, so that the concentrated light selectively heats and treats the target tissue.
